(19) United States
(12) Patent Application Publication
(10) Pub. No.: US 2007/0093867 A1
(43) Pub. Date: Apr. 26, 2007

(54) Acupressure Treatment Device

(76) Inventor: Ron Savoia, Granbury, TX (US)

Correspondence Address: QUICKPATENTS, INC., 31877 DEL OBISPO, SUITE 202, SAN JUAN CAPISTRANO, CA, 92675, US

(21) Appl. No.: 11/278,544

(22) Filed: Apr. 4, 2006

Related U.S. Application Data
(60) Provisional application No. 60/596,776, filed on Oct. 20, 2005.

(51) Int. Cl. A61B 17/00

(52) U.S. Cl. 606/204

(57) ABSTRACT

An acupressure clip for relieving a user's headache pain is disclosed. A generally U-shaped clip member includes an active arm and a support arm. Each arm meets at a spring portion, and each arm and spring portion include a continuous outside surface, a continuous inside surface, and at least one continuous edge surface. A pressure nub projects from the inside surface of the active arm and extends towards the supporting arm at a point closest to the supporting arm. In use, the clip is positioned on the user's hand between the thumb and the forefinger such that the pressure nub contacts and applies continuous pressure to the LI-4 acupressure point of the person's hand to relieve the person's headache pain. The clip is retained on the hand through friction and the spring force caused by separating the two arms, thus allowing uninhibited use of the hands.

### CROSS-REFERENCE TO RELATED APPLICATIONS

This application claims the benefit of U.S. Provisional Patent Application 60/596,776, filed on Oct. 20, 2005, and incorporated herein by reference.

### STATEMENT REGARDING FEDERALLY SPONSORED RESEARCH AND DEVELOPMENT

Not Applicable.

1. Field of the Invention

This invention relates to acupressure devices, and more particularly to an acupressure clip for the LI-4 acupressure point of a person's hand.

2. Discussion of Related Art

It is known in the art that headaches are often relieved by taking medications, such as ibuprofen, acetaminophen, aspirin, or the like. Some people, however, are physiologically intolerant to such medications, or for other reasons do not wish to ingest such medications. For such headache victims there are few practical pain relief solutions. Certain headaches, it has been found, may be relieved by applying a continuous pressure to a person's LI-4 acupressure point (the so-called Large Intestine 4 acupressure point). This point is located at the back of a person's hand, deep in the fleshy web between the forefinger and the thumb. Relieving a headache in this fashion precludes the need to take medication, and may therefore be much preferred. However, it has been previously difficult to apply pressure to this point for any extended period of time, particularly when faced with the need to perform other tasks simultaneously, such as typing, or the like.

One device, taught in U.S. Pat. No. 4,549,536 to Varjabedian on Oct. 29, 1985, teaches a spring-loaded pivoting two-part jaw that allows the user to apply pressure to the LI-4 acupressure point. While such a device may be suited to temporarily applying pressure to this acupressure point, it does have the drawback of having a relatively high profile and obtuse edges, making it easy to snag on objects inadvertently while using one's hands for other tasks. Moreover, such a device is biased to be normally open, such that the user must actively counter a spring in the device to apply pressure to the user's other hand. As such, the user is unable to do anything else with either of his hands while using such a device. Further, such a device is overly complicated in that it requires at least four separate pieces that need to be assembled. Consequently, such a device is relatively expensive to manufacture. Still further, such a device has a relatively sharp point that, over time, can cause bruising or otherwise damage the area around the LI-4 acupressure point.

U.S. Pat. No. 5,709,647 to Ferber on Jan. 20, 1998 teaches several devices spanning several different embodiments. One such embodiment, illustrated in FIG. 1B, illustrates a clip that presses against the LI-4 acupressure point around the web between a person's forefinger and thumb. Such a prior art device includes an adjustable knob that protrudes inconveniently from the top surface thereof, and is therefore easily snagged on objects inadvertently while using one's hands. Further, such a device is comprised of several distinct parts, and is therefore relatively expensive to manufacture. Still further, the adjustable knob can, over time, become loosened undesirably. Other embodiments taught in this patent have similar drawbacks.

Another relevant prior-art device is disclosed in U.S. Pat. No. 5,094,227 to Eglauf et al. on Mar. 10, 1992. Such a device has multiple parts and is incredibly cumbersome to use.

U.S. Pat. No. 4,320,760 to Sun et al. on Mar. 23, 1982 discloses another related prior-art device that is cumbersome to use. In use, the device forms a generally circular shape, which is prone to becoming dislodged from the intended acupressure point when bumped accidentally. Such a device is best suited for use on the human ear, for which it is designed, and is therefore not well-suited for use to relieve headaches using the LI-4 acupressure point.

U.S. Design Patent D487,930 to Amoroso on Mar. 30, 2004 is a hand-held device for applying pressure to an acupressure point, and is not well-suited for hands-free use. Therefore, there is a need for an inexpensive, preferably unitary device for applying pressure to the LI-4 acupressure point. Such a needed device would have no obtuse angles or other protrusions that could be easily snagged on objects while working with the hands. Further, such a needed device would remain on the user's hand by friction, and would not be required to be held in place by use of another hand. Such a needed device would allow quick visual observation of the area of the hand to which it is applied, without removal of the device. The present invention accomplishes these objectives.

### SUMMARY OF THE INVENTION

The present device is an acupressure clip for relieving headaches in a person having a hand with a thumb, forefinger, and an LI-4 acupressure point on the back thereof A generally U-shaped clip member includes an active arm and a support arm. Each arm meets at a spring portion, and each arm and spring portion include a continuous outside surface, a continuous inside surface, and at least one continuous edge surface. A pressure nub projects from the inside surface of the active arm and extends towards the supporting arm at a point closest to the supporting arm.

Preferably each arm flares away from the other arm at a distal end thereof. Further, the distal ends of each arm are preferably rounded such that the clip has exactly one smooth, continuous edge surface. As such, the clip does not have any corners that may snag objects that are inadvertently brought into contact with the clip while the person wears the clip and performs other tasks with his hands.

In use, the clip is positioned on the hand between the thumb and the forefinger such that the pressure nub contacts and applies continuous pressure to, the LI-4 acupressure point of the person's hand to relieve the person's headache pain. The clip may be slid onto the hand easily with a web of the hand between the forefinger and the thumb acting to contact and apply a separating pressure against each arm to facilitate placement of the pressure nub over the LI-4 acupressure point of the person's hand.

The present invention is an inexpensive, single-part device for applying pressure to the LI-4 acupressure point of a user's hand. The present device has no obtuse angles or other protrusions that can be easily snagged on objects while working with the hands, thereby allowing the user to continue working with his hands while the device is being worn. Further, the present invention remains on the user's hand by friction, and does not require holding in place by use of another hand or complicated retaining mechanism. The present invention allows quick visual observation of the area of the hand to which it is applied, without removal of the device. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the invention;

FIG. 2 is a left-side elevational view of the invention;

FIG. 3 is a right-side elevational view of the invention;

FIG. 4 is a top plan view of the invention;

FIG. 5 is a bottom plan view of the invention;

FIG. 6 is a rear elevational view of the invention;

FIG. 7 is a front elevational view of the invention;

FIG. 8 is a perspective view of the invention, illustrating a clip of the invention on a person's hand; and

FIG. 9 is a perspective view of the invention, illustrating the clip as removed from the person's hand.

### DETAILED DESCRIPTION OF THE PREFERRED EMBODIMENT

FIGS. 1, 8, and 9 illustrate an acupressure clip 10 for relieving headaches in a person having a hand 20 with a thumb 22, forefinger 24, and an LI-4 acupressure point 23 on the back 25 thereof. A generally U-shaped clip member 30 includes an active arm 40 and a support arm 50. Each arm 40,50 meets at a rounded spring portion 60, and each arm 40,50 and spring portion 60 include a continuous outside surface 70, a continuous inside surface 80, and at least one continuous edge surface 90. The corners between all surfaces 40,50,60 are preferably rounded so as to prevent inadvertent snags with other objects. The spring portion 60 biases the arms 40,50 together when the arms 40,50 are separated from each other.

A pressure nub 100 projects from the inside surface 80 of the active arm 40 and extends towards the supporting arm 50 at a point 110 closest to the supporting arm 50 (FIG. 3). The pressure nub 100 is preferably a rounded dome shape, but may also be, in an alternate embodiment, a four-sided pyramid shape, a three-sided pyramid shape, an oval shape, or any other suitable shape for contacting the person's LI-4 pressure point 23.

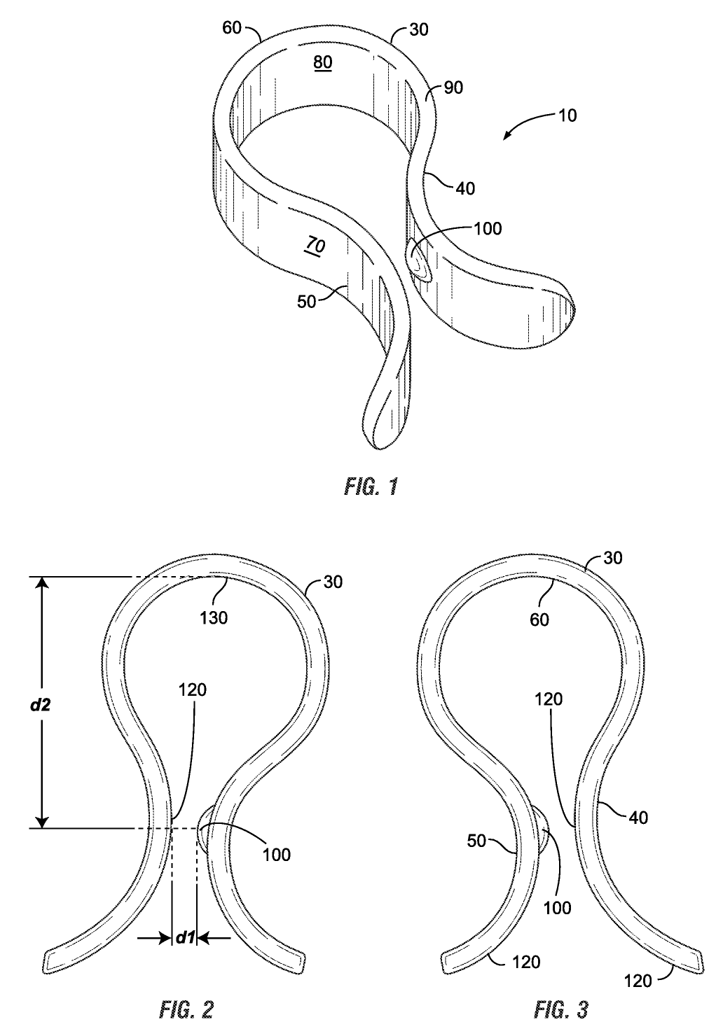

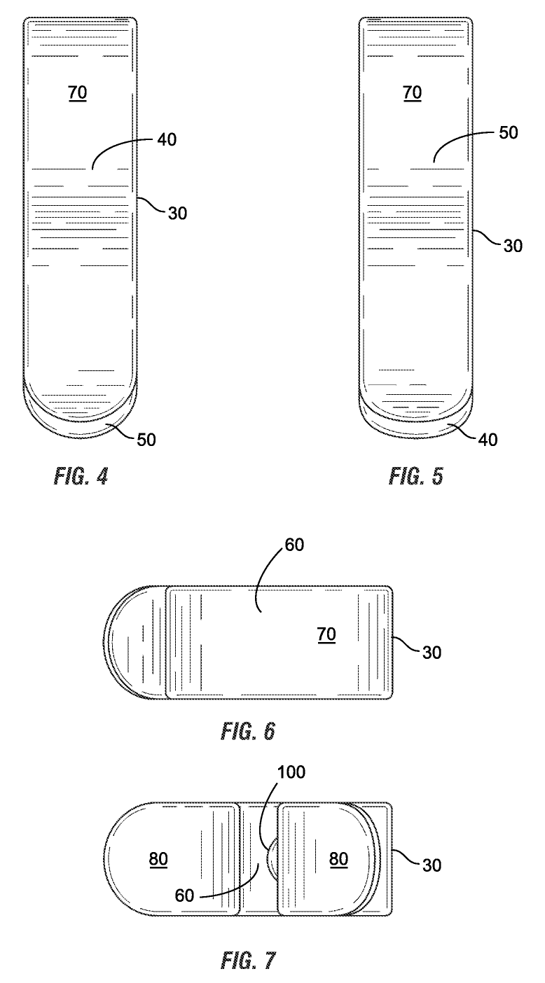

Preferably each arm 40,50 flares away from the other arm 50,40 at a distal end 120 thereof (FIGS. 2, 3 and 7). Further, the distal ends 120 of each arm 40,50 are preferably rounded such that the clip 10 has exactly one smooth, continuous edge surface 90. As such, the clip 10 does not have any corners that may snag objects that are inadvertently brought into contact with the clip 10 while the person wears the clip 10 and performs other tasks with his hands 20. Preferably the clip 10 is made from a rigid plastic material, although the clip 10 could be made from any other suitably rigid material that applies a spring force between the arms 40,50 when the arms 40,50 are separated, such as metal, wood, or the like.

A distance d1 between the pressure nub 100 and the support arm 40 is preferably between one and three millimeters when not engaged with the person's hand 20 (FIG. 2). However, the distance d1 could be between zero and four millimeters in alternate embodiments. Moreover, a distance d2 between the pressure nub 100 and a point 130 on the inside surface 80 of the clip member 30 furthest from the nub 100 is between approximately two and three centimeters, and preferably approximately 27 millimeters. The thickness of the clip 10 is preferably uniform and not greater than about 2.5 millimeters so as to keep the clip 10 close to the hand 20 while the person uses the hand 20 for other tasks while wearing the clip 10, preventing inadvertent snags. Further, the width of each arm 40,50 and the spring portion 60 is preferably uniform and between ten and fourteen millimeters.

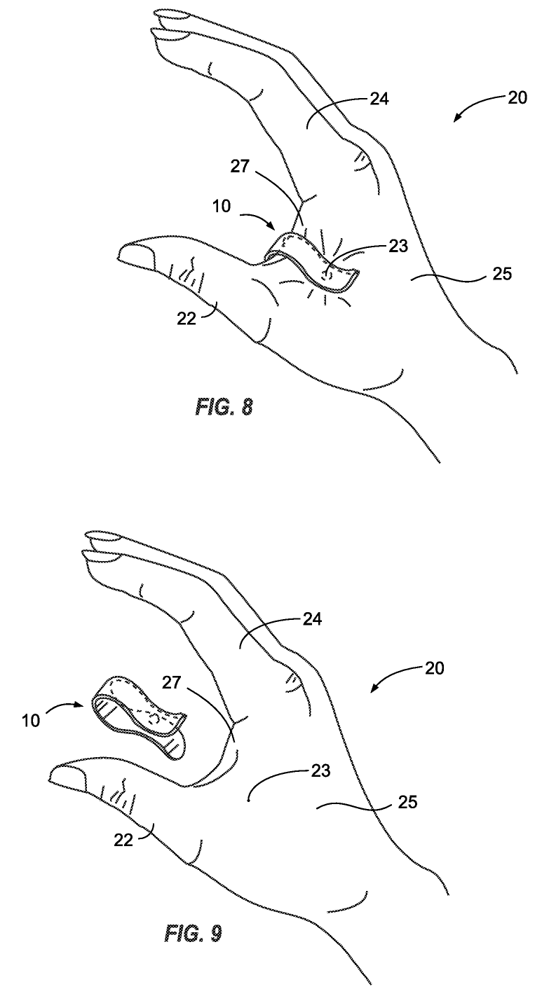

In use, the clip 10 is positioned on the hand 20 between the thumb 22 and the forefinger 24 such that the pressure nub 100 contacts and applies continuous pressure to, the LI-4 acupressure point 23 of the person's hand 20 to relieve the person's headache pain. The clip 10 may be slid onto the hand 20 easily with a web 27 of the hand between the forefinger 24 and the thumb 22 acting to contact and apply a separating pressure against each arm 40,50 to facilitate placement of the pressure nub 100 over the LI-4 acupressure point 23 of the person's hand 20 (FIGS. 8 and 9). In an embodiment wherein the clip 10 is substantially transparent or translucent, the person may observe the nub 100 pressing against his LI-4 acupressure point 23 to ensure that a bruise or other damage isn't caused by the clip 10. In an embodiment wherein the clip 10 is opaque, the clip 10 may be decorated with decals, marking pens, or otherwise colored or decorated to the person's taste.

While a particular form of the invention has been illustrated and described, it will be apparent that various modifications can be made without departing from the spirit and scope of the invention. For example, the distal ends 120 may be squared-off, or the specific dimensions of d1 and d2 may be adjusted slightly to accommodate person's having either smaller or larger hands 20. Accordingly, it is not intended that the invention be limited, except as by the appended claims.

What is claimed is:

1. An acupressure clip for relieving headaches in a person, the person having a hand with a thumb and forefinger and an LI-4 acupressure point on the back thereof, the acupressure clip comprising:
a generally U-shaped clip member having an active arm and a supporting arm, each arm meeting at a spring portion, each arm and the spring portion including a continuous outside surface, a continuous inside surface, and at least one continuous edge surface; and
a pressure nub projecting from the inside surface of the active arm and extending towards the supporting arm at a point closest to the supporting arm;
whereby with the clip positioned on the hand of the person between the thumb and forefinger, the pressure nub applies continuous pressure to the LI-4 acupressure point of the person's hand to relieve headache pain.

2. The acupressure clip of claim 1 wherein each arm flares away from the other arm at a distal end thereof, whereby the clip may be slid onto the hand easily with a web of the hand between the forefinger and the thumb acting to contact and apply a separating pressure against each arm to facilitate placement of the pressure nub over the LI-4 acupressure point of the person's hand.

3. The acupressure clip of claim 1 wherein the pressure nub is generally rounded dome shape.

4. The acupressure clip of claim 1 wherein the pressure nub is generally a four-sided pyramid shape.

5. The acupressure clip of claim 1 wherein a distance d1 between the pressure nub and the supporting arm is between approximately one and three millimeters.

6. The acupressure clip of claim 5 wherein the distance d1 between the pressure nub and the supporting arm is approximately two millimeters.

7. The acupressure clip of claim 1 wherein a distance d2 between the pressure nub and a point on the inside surface of the clip member furthest from the nub is between approximately two and three centimeters.

8. The acupressure clip of claim 7 wherein the distance d2 between the pressure nub and the point on the inside surface of the clip member furthest from the nub is approximately twenty-seven millimeters.

9. The acupressure clip of claim 2 wherein the distal ends of each arm are rounded such that the clip member has exactly one continuous edge surface.

10. The acupressure clip of claim 1 wherein the clip member is substantially transparent, whereby the condition of the hand at the LI-4 acupressure point may be observed through the clip while the clip is engaged to the person's hand.

11. The acupressure clip of claim 1 wherein the clip member is substantially translucent.

12. The acupressure clip of claim 1 wherein the clip member is opaque.
